(19) United States
(12) Patent Application Publication
(10) Pub. No.: US 2021/0166782 A1
(43) Pub. Date: Jun. 3, 2021

(54) CLINICAL INTERPRETATION OF GENOMIC AND TRANSCRIPTOMIC DATA AT THE POINT OF CARE FOR PRECISION CANCER MEDICINE

(71) Applicant: Dana-Farber Cancer Institute, Inc., Boston, MA (US)

(72) Inventors: Eliezer Van Allen, Brookline, MA (US); Brendan Reardon, Brookline, MA (US); Nathaniel Moore, Scottsdale, AZ (US)

(21) Appl. No.: 17/047,325

(22) PCT Filed: Apr. 12, 2019
(86) PCT No.: PCT/US19/27338

§ 371 Date: Oct. 13, 2020

Related U.S. Application Data
(60) Provisional application No. 62/656,778, filed on Apr. 12, 2018.

(51) Int. Cl. G16B 20/10; G16H 10/40; G16H 10/60; G16H 50/70; G16H 70/60; G16H 50/30; G16B 20/00

(52) U.S. Cl. CPC G16B 20/10; G16H 10/40; G16H 10/60; G16H 20/10; G16H 70/60; G16H 50/30; G16B 20/00; G16H 50/70

(57) ABSTRACT

Feature-based clinical interpretation of whole exome and transcriptome data for precision cancer medicine is provided. In various embodiments, genomic data of a subject is received. The genomic data comprises somatic mutations. A plurality of features is determined from the genomic data of the subject. A similarity metric is determined between the plurality of features and each of a plurality of reference genomes. One or more potentially actionable feature is determined from the similarity.

### CROSS-REFERENCE TO RELATED APPLICATIONS

This application claims the benefit of U.S. Provisional Application No. 62/656,778, filed Apr. 12, 2018, which is hereby incorporated by reference in its entirety.

### STATEMENT REGARDING FEDERALLY SPONSORED RESEARCH

This invention was made with Government support under Grant No. K08CA188615 awarded by the National Institutes of Health. The Government has certain rights to this invention.

### BACKGROUND

Embodiments of the present disclosure relate to feature-based clinical interpretation of whole exome and transcriptome data for precision cancer medicine, and more specifically, to methodology for performing clinical interpretation of genomic and transcriptomic data at the point of care for precision cancer medicine.

### BRIEF SUMMARY

In various embodiments, systems, methods, and computer program products are provided for feature-based clinical interpretation of genomic data. Genomic data of a subject is received. The genomic data comprises somatic mutations. A plurality of features is determined from the genomic data of the subject. A similarity metric is determined between the plurality of features and each of a plurality of reference genomes. One or more potentially actionable feature is determined from the similarity.

In some embodiments, the genomic data of the subject further comprise germline mutations. In some embodiments, the genomic data of the subject further comprise copy number alterations. In some embodiments, the genomic data of the subject further comprise fusions.

In some embodiments, an associated score is determined for the one or more potentially actionable feature, the score being indicative of support for a clinical action.

In some embodiments, the reference genomes comprise the Cancer Genome Atlas (TCGA).

In some embodiments, the similarity metric comprises a distance within a vector space between a vector corresponding to the plurality of features and vectors corresponding to the plurality of reference genomes. In some embodiments, the distance comprises a Euclidian distance. In some embodiments, the distance comprises a cosine distance. In some embodiments, the distance comprises a Jaccard similarity.

In some embodiments, the plurality of features comprise somatic-germline overlap, DNA-RNA overlap, mutational burden, MSI status, and/or connections.

In some embodiments, the genomic data of the subject is received at a point of care.

### BRIEF DESCRIPTION OF THE SEVERAL VIEWS OF THE DRAWINGS

FIG. 1 illustrates the Precision Heuristics for Interpreting the Alteration Landscape (PHIAL) system.

FIG. 2 illustrates the overall workflow of PHIAL.

FIG. 3 illustrates the Tumor Alterations Relevant for Genomics-Driven Therapy (TARGET) database is illustrated.

FIG. 4 illustrates an exemplary workflow according to embodiments of the present disclosure.

FIG. 5 illustrates evaluation of DNA-RNA overlap according to embodiments of the present disclosure.

FIG. 6 illustrates exemplary report data according to embodiments of the present disclosure.

FIG. 7 illustrates the percentage of samples with at least one putatively actionable SNV, InDel, or CNV across exemplary TCGA studies by the PHIAL-TARGET approach.

FIGS. 8A-B illustrates the workflow of a molecular oncology almanac according to embodiments of the present disclosure.

FIGS. 9A-F illustrate results of an exemplary molecular oncology almanac according to embodiments of the present disclosure.

FIG. 10 illustrates an example of Euclidian distance to compute similarity.

FIG. 11 illustrates an example of cosine distance to compute similarity.

FIG. 12 illustrates an example of Jaccard similarity to compute similarity.

FIG. 13 illustrates a method of feature-based clinical interpretation of genomic data according to embodiments of the present disclosure.

FIG. 14 depicts a computing node according to embodiments of the present disclosure.

### DETAILED DESCRIPTION

Cancer treatment has been revolutionized by the ability to obtain genomic sequence and other molecular data from an individual patient's tumor. This has led to a massive increase in the quantity of data available to clinicians—an increase with therapeutic implications if interpreted well.

A given patient may have tens to thousands of single nucleotide variants, up to thousands of insertions or deletions, tens to thousands of copy number alterations, and up to thousands of rearrangements. This poses a challenge as to how variants should be prioritized for functional validation. Alternatives include

Cancer researchers would benefit from a standard method of interpreting putative actionability from a patient's many types of genomic data. To address this need, the present disclosure provides a molecular oncology almanac for integrative clinical interpretation of molecular profiles to guide precision cancer medicine. In various embodiments, a database of relationships between genetic alterations and potential clinical actions is provided.

Alternative methods to perform clinical interpretation are typically limited in multiple ways:
1) They only focus on DNA based information
2) They only focus on a subset of genes within the genome
3) They cannot consider multiple interacting events within a patient's tumor
4) They cannot incorporate fusion data from RNA sequencing
5) They have limited points of entry
6) They have no mechanism to link patient data to preclinical models systematically in order to refine predictions about actionability

The methods herein overcome each of these limitations of alternative approaches.

Clinical interpretation algorithms and knowledge bases (e.g., PHIAL, OncoKB) may be used for clinical decision making. These approaches are generally limited to first order genomic relationships (e.g., BRAFV600E & RAF/MEK inhibition). The increasing complexity of molecular data generated at the point of care, including whole exome and transcriptome results, along with the expanded therapeutic landscape in cancer and expanded preclinical model systems with matching data, necessitate novel algorithms to enable robust and modern clinical interpretation of a cancer patient's molecular data to accelerate precision cancer medicine. The present disclosure provides a paired feature-based clinical interpretation algorithm and knowledge system for cancer genomic and transcriptomic data to inform treatment decisions at the point of care and provide researchers with rapid assessment of tumor actionability.

Various methods according to the present disclosure expand upon PHIAL to predict actionability based on first-order genomics using SNVs (from both whole-exome sequencing and bulk RNA-seq), InDels, SCNAs, and fusions to further infer global features of an individual tumor such as mutational burden, mutational signature profile, MSI-status, somatic-germline interaction, and connections between events. Predictive implication values are assigned to reflect the validities of the database's drug sensitivity, resistance, and prognostic claims. Individual tumors profiles are also matched to similar preclinical systems that have functional assessments for further refinement of actionability scores based on observed putative clinical actionability.

The feature-based approach is benchmarked against the PHIAL & TARGET methodology across two cohorts that include both whole exome and transcriptomic data—150 castrate resistant prostate cancers and 110 metastatic melanomas. PHIAL identified 1281 putatively actionable or biologically relevant alterations, with a median of 3 events per patient and 94% of patients having at least 1 event. The feature-based approach identified 1767 putatively actionable or biologically relevant variants or features, with a median of 5 events per patient and 97% of patients having at least 1 event. Of the these patients, 27% had at least 1 variant associated with an FDA-approved therapy and 18% had events associated with a clinical trial. It also identified that 29% of samples had a putatively actionable global feature.

Thus, DNA and RNA based interpretation method is able to identify and rank more putatively actionable first-order genomic alterations than PHIAL & TARGET, while also providing insight to global features of individual tumors. Increased accessibility of clinical interpretation through cloud-based web portals and genomic reports may aid in sample contextualization, especially at the point of care.

These methods are useful for diagnostic testing laboratories as part of their product development, pharmaceutical companies for companion diagnostics with therapeutics, and electronic health record companies as part of their genomic solutions.

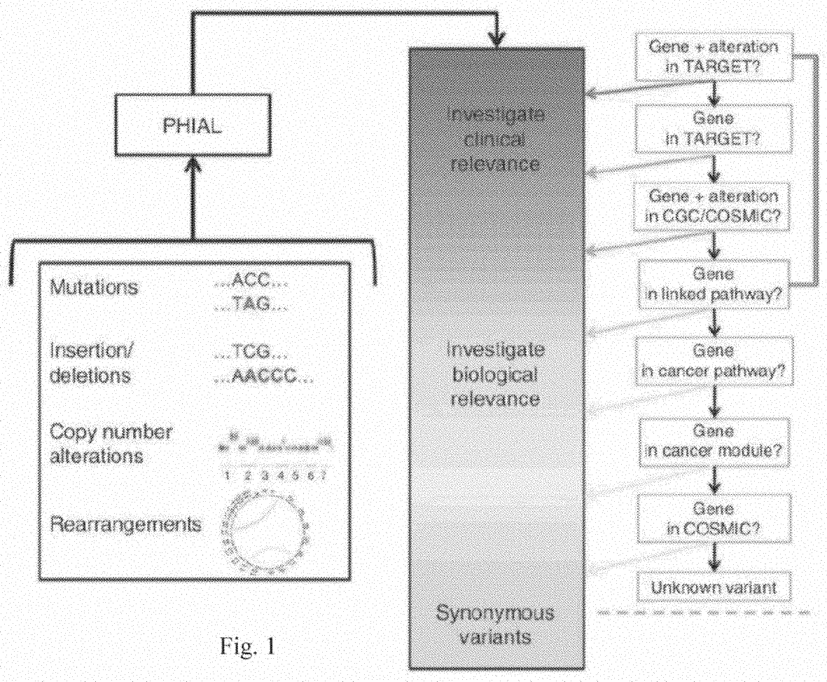

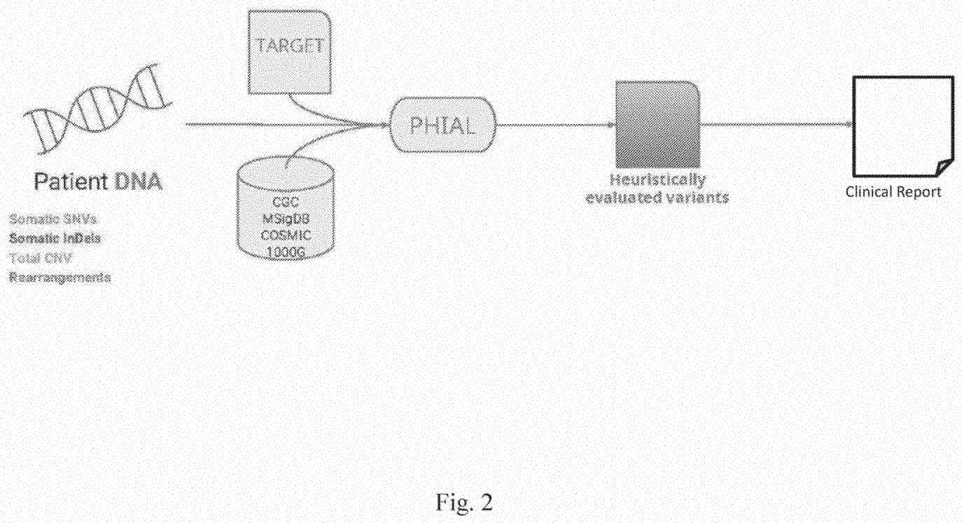

Referring to FIG. 1, the Precision Heuristics for Interpreting the Alteration Landscape (PHIAL) system is illustrated. PHIAL is a heuristic-based clinical interpretation algorithm that sorts somatic variants by clinical and biological relevance. The overall workflow of PHIAL is illustrated in FIG. 2.

PHIAL provides rapid assessment of diverse patient tumor data (˜5-10 min run time). It provides interactive patient actionability reports, and intuitive visualization of scored variants. Moreover, it is approved by the Clinical Laboratory Improvement Amendments (CLIA).

However, PHIAL is limited to characterizing only first-order and gene-level genomic relationships. It is very dependent on upstream annotation and formatting. It only considers alterations from whole-exome sequencing of DNA. It has limited code coverage, and reports are dependent on supporting files and are thus not portable.

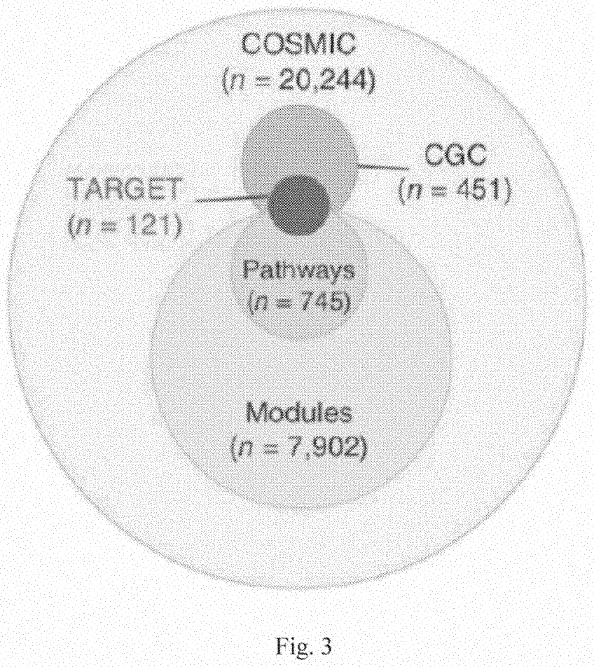

Referring to FIG. 3, the Tumor Alterations Relevant for Genomics-Driven Therapy (TARGET) database is illustrated. TARGET is a database of genes that may have therapeutic, prognostic, and diagnostic implications for patients with cancer.

TARGET represents the first effort to widely catalogue alteration-action assertions that are clinically relevant to oncology. It is portable and easily distributable. TARGET enables rapid assessment of alterations for putative actionability.

However, TARGET contains outdated assertions, no citations, and is not stored in a scalable architecture. It is limited to gene and alteration type relationships.

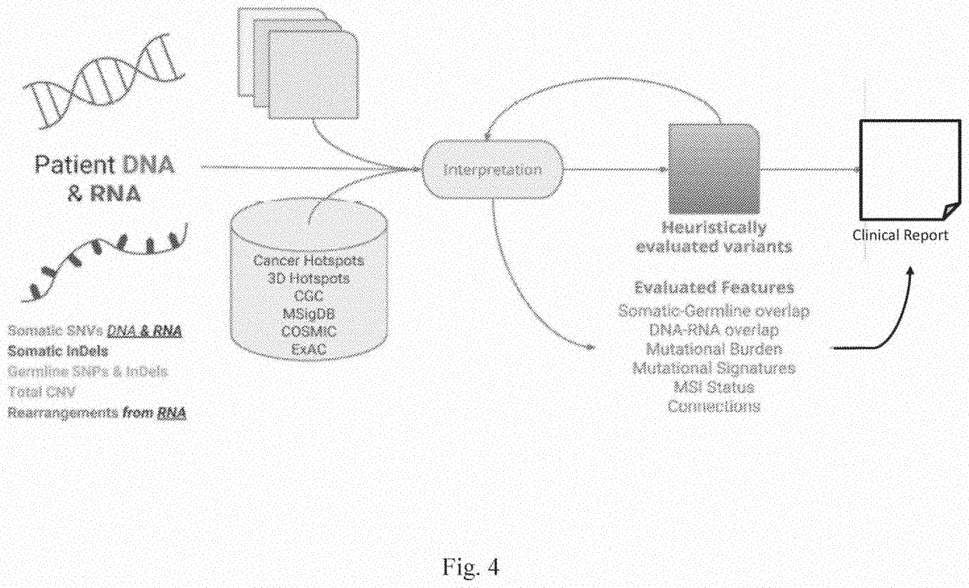

Referring to FIG. 4, an exemplary workflow according to embodiments of the present disclosure is illustrated. In various embodiments, various general heuristics are applied, including identification of desired variants. Such heuristics include whether a given gene is in: an almanac; a cancer hotspot, a 3D cancer hotspot, the Cancer Genome Censuc (CGS), the same pathway as a SALSA gene, an MSigDB cancer pathway, an MSigDB cancer module, COSMIC, or is a variant of uncertain significance (VUS). In addition, items are of particular interest where features and/or alteration match.

In some embodiments, only certain kinds of alterations are accepted. For example, SNVs and InDels (e.g., Missense, Nonsense, Nonstop, Frameshift, lndels), Copy Number (e.g., Amplitude ≥97.5 percentile or ≤2.50 percentile segment mean), or Fusion (e.g., Segment Fragments ≥5).

In various embodiments, a scoring rubric is applied to alterations as set forth below in Table 1.

TABLE 1

| | |
|---|---|
| FDA-Approved | Validated association between the alteration and an |
| | FDA-approved clinical action. (E.g. FDA approved |
| | relationship) |
| Guideline | Association between alteration and clinical action is |
| | standard of care. |
| Clinical trial | Alteration is or has been used as an eligibility |
| | criterion for a clinical trial. (E.g. Enrollment |
| | criteria for a clinical trial) |
| Clinical evidence | Early clinical evidence supports the alteration-action |
| | relationship. (E.g. A published study in humans) |
| Preclinical evidence | Preclinical evidence supports the alteration-action |
| | relationship. (E.g. A published study in mice or cell |
| | lines) |
| Inferential | Inferential evidence supports the alteration-action |
| | relationship. (E.g. A simulation or mathematical |
| | model, such as a mutational signature) |

In various embodiments, features are evaluated including: Somatic-Germline overlap, DNA-RNA overlap, Mutational Burden, Mutational Signatures, MSI Status, and Connections.

In evaluating Germline-Somatic Interaction, consider an example Somatic observation: TP53 c.818G>A, p.R273H. If the gene is also altered in the germline, the variant prioritization is increasesd (Nonsense, Splice Site, Frameshift, Indel variants only). If the alteration is common in ExAC (e.g., > 1/1,000 alleles), variant prioritization is decreased. In addition, in some embodiments, additional factors are considered, including somatic variants that have germ line variants in the same gene, germline variants that have somatic variants in the same gene, pertinent negatives, germline variants in a Cancer-related genes that are rarer than 1/1,000 alleles in ExAC, and incidental findings that appear in the American College of Medical Genetics and Genomics.

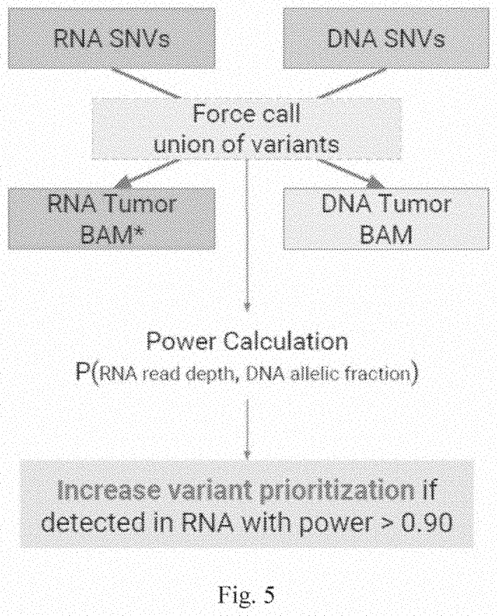

Referring to FIG. 5, in various embodiments, DNA-RNA overlap is evaluated. As pictured, variant prioritization is increased if detected in RNA with power >0.90.

In various embodiments, (Nonsyn) Mutational Burden is evaluated. This provides an initial similarity measure between cancers. A patient's nonsyn mutational burden relative to its percentile relative to TCGA and TCGA tissue type is provided in some embodiments. The mutational burden is flagged if >80th percentile within tissue type and >10 mutations per Mb.

In various embodiments, mutational signatures are evaluated. Mutational signatures are characteristic combinations of mutation types arising from specific mutagenesis processes such as DNA replication infidelity, exogenous and endogenous genotoxins exposures, defective DNA repair pathways and DNA enzymatic editing. Various methods are known in the art for computing mutational signatures.

In various embodiments, Microsatellite Instability is evaluated. Microsatellite instability (MSI) is the condition of genetic hypermutability (predisposition to mutation) that results from impaired DNA mismatch repair (MMR). The presence of MSI represents phenotypic evidence that MMR is not functioning normally. In particular, MSI is flagged in various embodiments where mutations are present in MSI genes (MSH2, PMS2, MSH6, POLE, MLH1, POLE2, ACVR2A, RNF43, JAK1, MSH3, ESRP1, PRDM2, DOCK3). In various embodiments only Nonsense, Splice Site, Frameshift, and Indel variants are considered.

In various embodiments, connections are evaluated. In particular, prioritization is increased where related events are reported, for example, Mutation POLE+COSMIC Signature 10, Mutation in ERCC2+COSMIC Signature 5, or Mutation in MSI Gene+COSMIC Signatures 6/15/20/21/26.

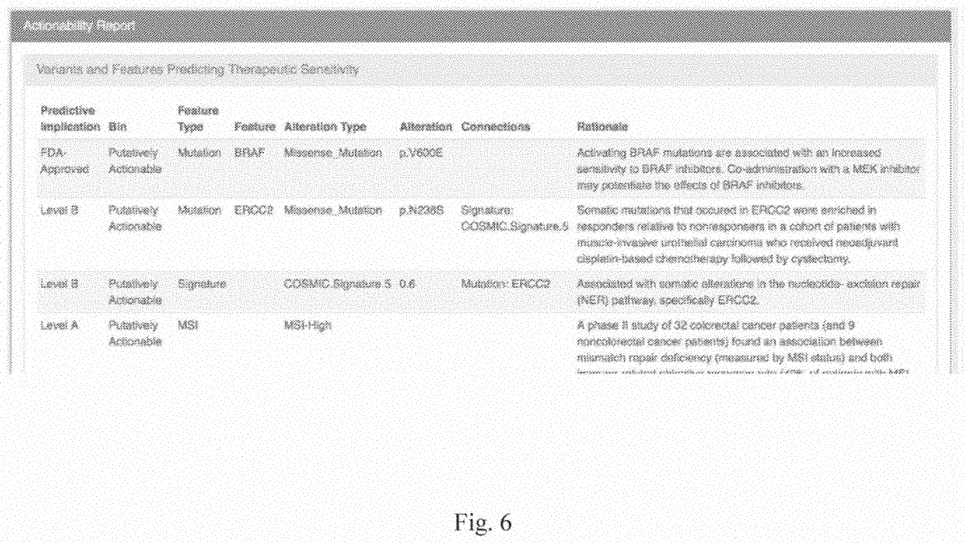

In various embodiments a patient actionability report is generated. In various embodiments, separate reporting of sensitive, resistance, prognostic, and biologically relevant relationships is provided. Various formats may be provided, including a portable html file. In various embodiments, easily readable assertion rationales are provided, including a link to a direct citation. Various user interface features may be provided to ease interpretation, such as an icon to indicate confidence in an alteration (e.g., to warn of low allelic fraction), or a detailed report with plots of additional metrics such as distribution of allelic fraction or mutational burden relatice to TCGA. Exemplary report data are provided in FIG. 6.

In various embodiments, a cloud-based web portal is provided for processing patient data and generating a report such as depicted in FIG. 6. In various embodiments, the cloud-based system is configured to provide a dedicated private instance of the analytic package in order to ensure the privacy of uploaded data.

In various embodiments, an alteration-action database is provided. In some embodiments, web-based database management is provided. In various embodiments, automate literature review is provided (for example, via Google Scholar and PubMed APIs). In various embodiments, this functionality may be provided through a web browser extension. In various embodiments, a user may flag an assertion if they think it is outdated or incorrect.

In various embodiments, a clinical interpretation algorithm is provided. In some embodiments, this algorithm incorporates allelic copy number, incorporate RNA expression and identifies concordance with copy number, and improves MSI and Connections features. In some embodiments, a detailed technical report is provided with additional data visualization.

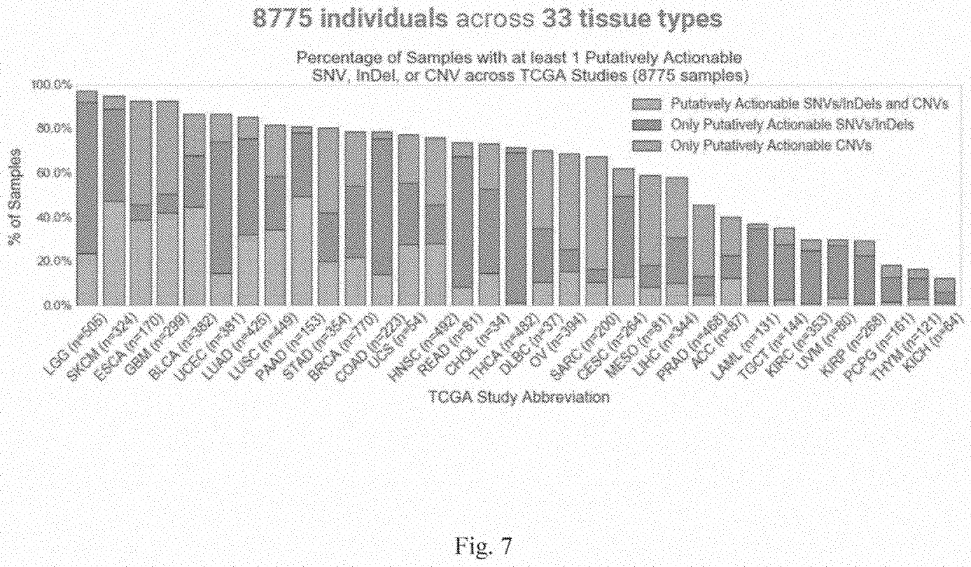

FIG. 7 shows the percentage of samples with at least one putatively actionable SNV, InDel, or CNV across exemplary TCGA studies (8775 samples) as designated by the PHIAL-TARGET approach. 69.9% of all samples contained at least one putatively actionable alteration. 83.4% of variants had at least one putatively actionable event or at least one biologically relevant alteration.

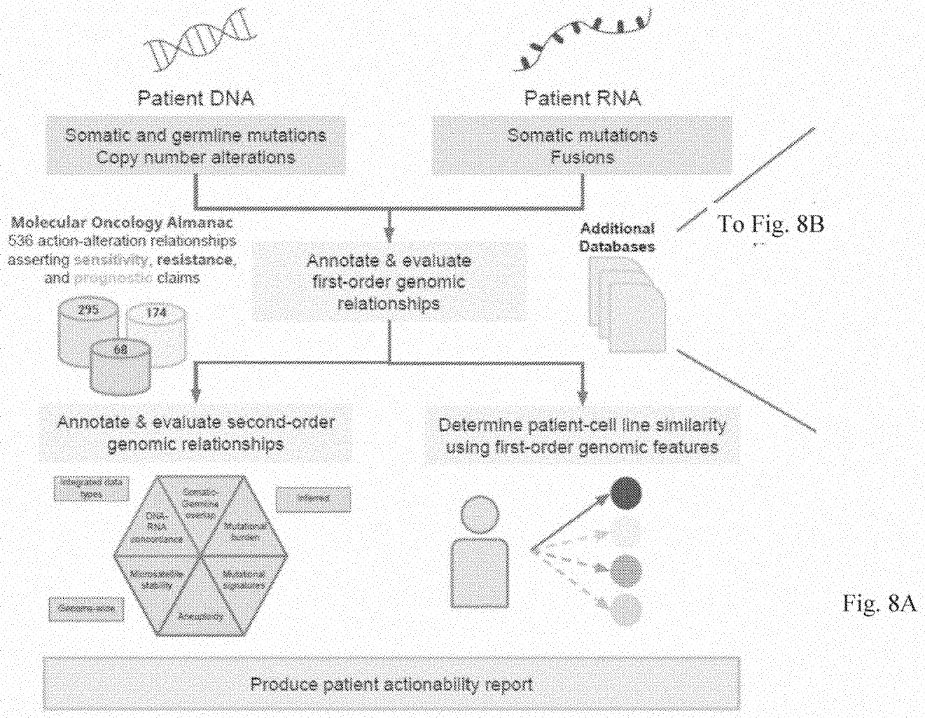

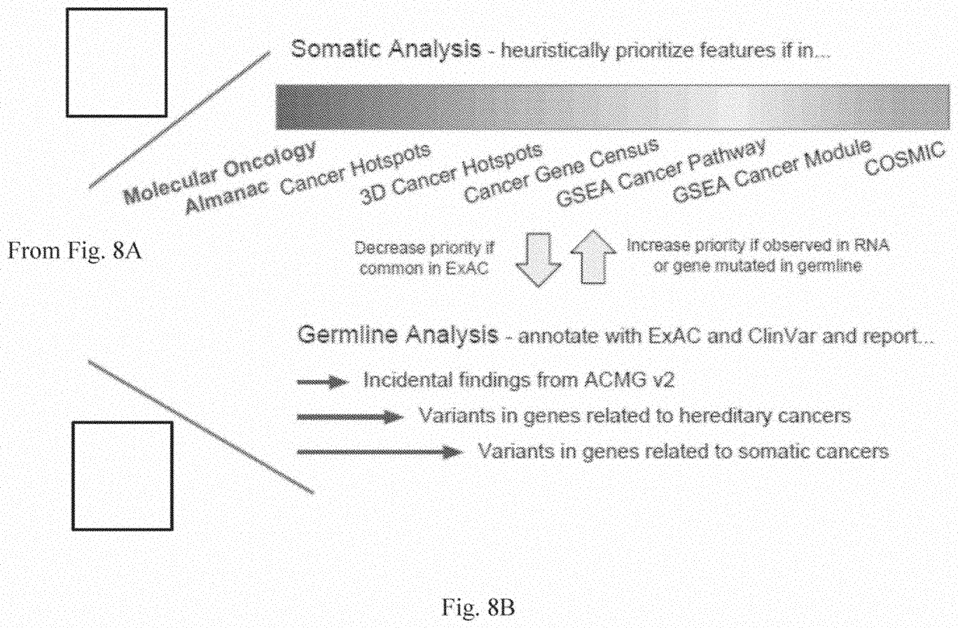

Referring now to FIGS. 8A-B, the workflow of a molecular oncology almanac according to embodiments of the present disclosure is illustrated. Whole-exome and transcriptome sequencing data can be leveraged to heuristically identify first-order genomic relationships associated with clinical action and their presence of variants in other databases. Furthermore, second-order relationships are evaluated and therapies based on genomic similarity to cell lines are reported.

The resulting molecular oncology almanac expands upon TARGET by adding 465 alteration-action relationships, bringing the total to 619; specifying predictive implications as sensitivity, resistance, or prognostic claims; creating a web portal to enable convenient access of a curated database and a web browser extension to facilitate community contributions.

The molecular oncology almanac interprets various sources of patient genomic data, including germline and RNA variants and fusions; reduces reliance upon upstream annotation; infers both first-order and second-order relationships (e.g., microsatellite instability and mutational signatures); and simplifies patient actionability reports.

In various embodiments, a cloud-based web portal is provided to allow users to identify putatively actionable and biologically relevant tumor variants and features. In various embodiments, a curated action-alteration database is provided for searching, containing assertions ranging from FDA-approved therapies to preclinical inferences. The clinical interpretation algorithm and alteration-action database enables rapid assessment of putative variant actionability for clinicians and aids in sample contextualization for researchers.

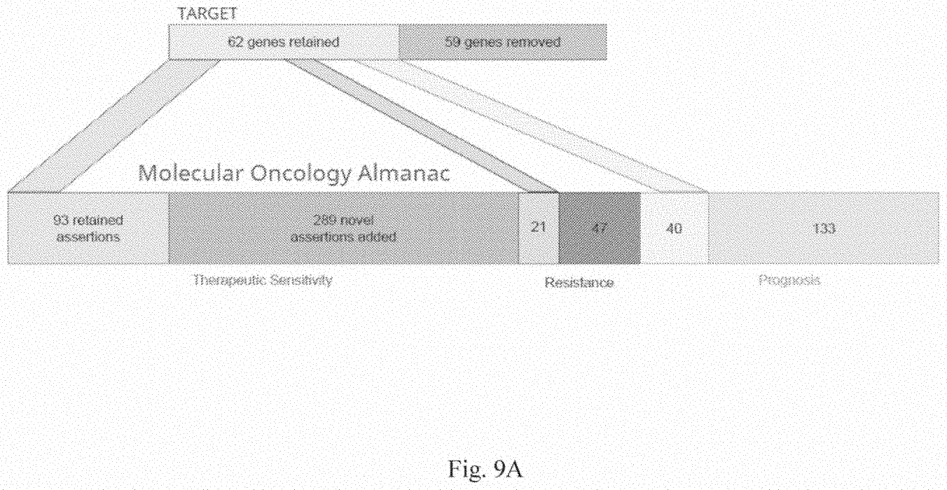

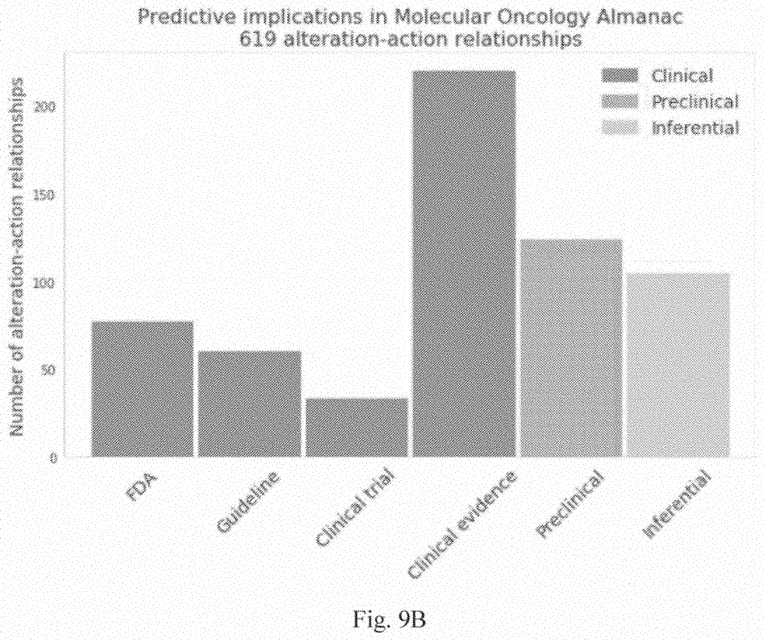

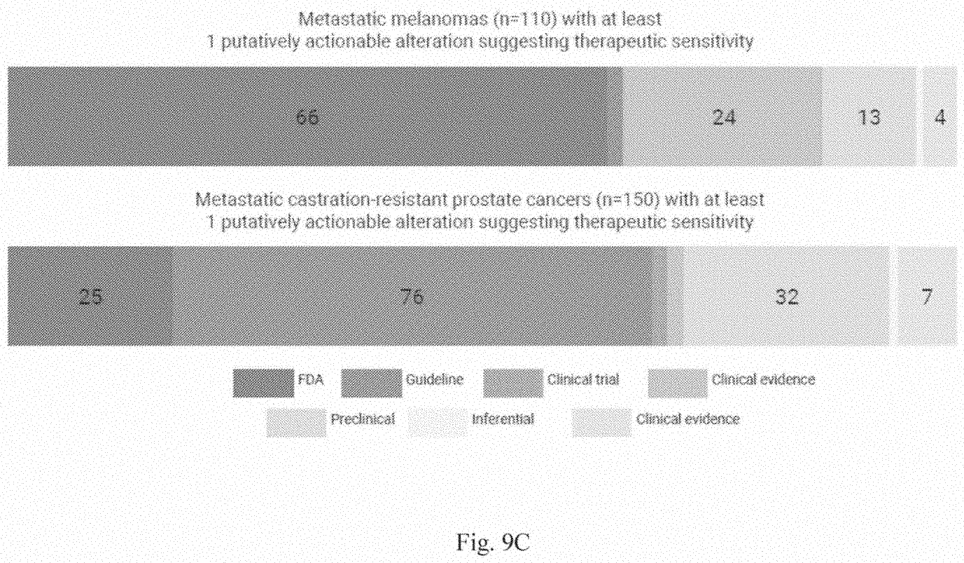

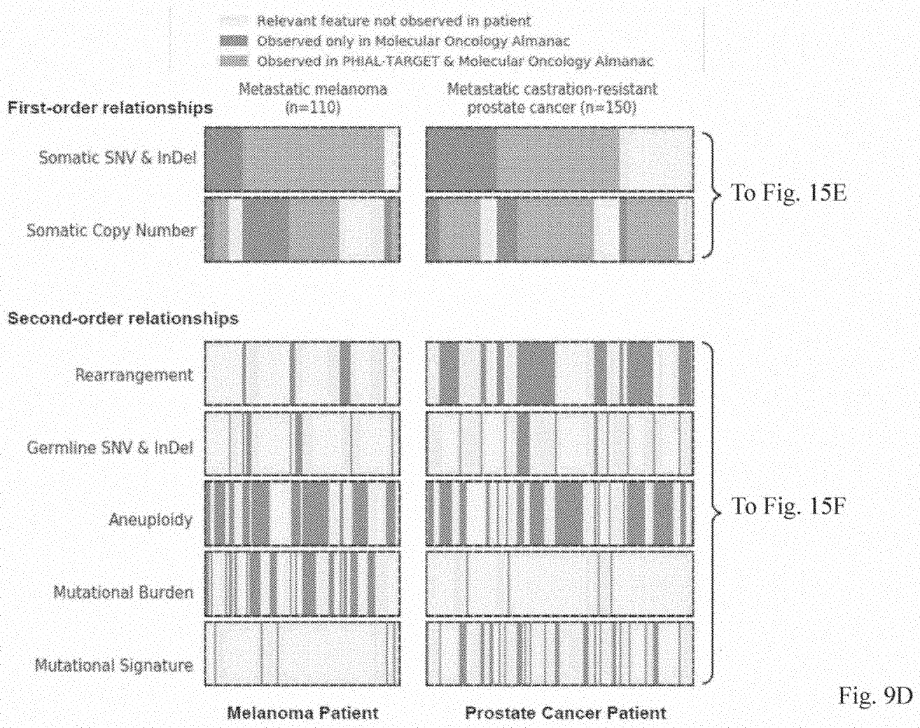

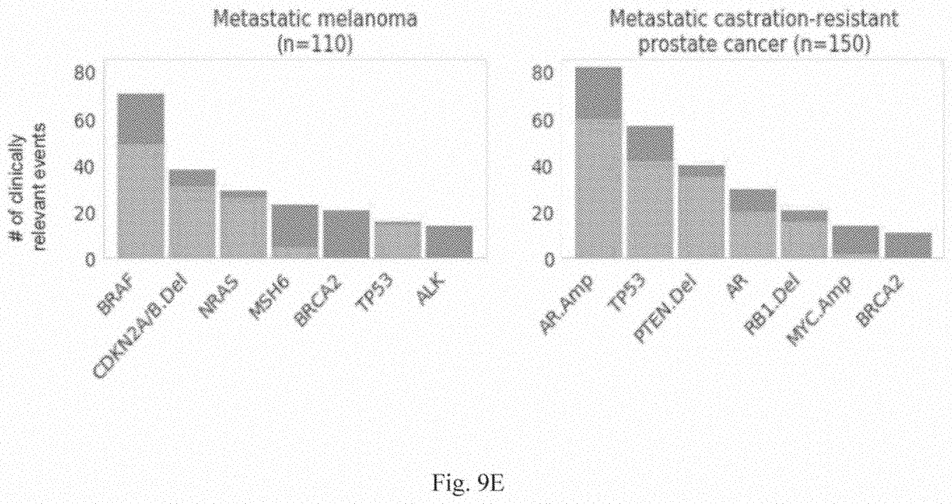

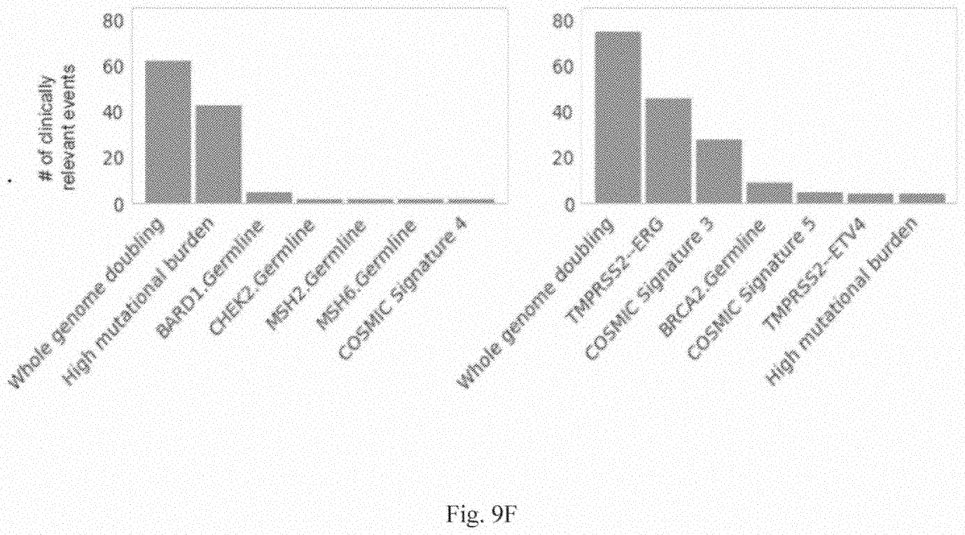

Referring to FIGS. 9A-F, the results of PHIAL and TARGET are compared to those of the Molecular Oncology Almanac using a 260 patient cohort consisting of both whole exome and transcriptome sequencing data (110 metastatic melanoma and 150 castration-resistant prostate cancer patients). The Molecular Oncology Almanac associated 17% of all putatively actionable relationships with an FDA-approved therapy, and 13% with a guideline or clinical trial.

FIG. 9A shows that the Molecular Oncology Almanac partly derives alteration-action relationships from the gene-centric TARGET. These relationships are represented as the lighter-color segment in each relationship category block. FIG. 9B shows predictive implications in the database ranging from FDA-approved to preclinical and inferential relationships. FIG. 9C illustrates an example in which the Molecular Oncology Almanac was applied to 110 metastatic melanoma and 150 castration-resistant prostate cancer patients. This shows a total of 2294 action-alteration relationships from 1604 features across all predictive implication levels, where at least the gene name and feature type matched a catalogued assertion. Considering only sensitive relationships, the highest predictive implication level is observed per patient. FIGS. 9D-F compares PHIAL-TARGET to the Molecular Oncology Almanac. More somatic nucleotide and copy number variants are observed, and additionally fusions, germline variants, aneuploidy, mutational burden, and mutational signatures are interpreted.

As compared to PHIAL and TARGET, the Molecular Oncology Almanac has improved the ability to identify and annotate putatively actionable genomic alterations in patient tumor samples, while also enabling characterization of higher-order molecular features by integrating multiple types of sequencing data. Expanding evidence sources to include preclinical and inferential studies reveals additional putatively actionable relationships. Additionally, these tools are accessible through the use of web portals and API endpoints, expanding the clinical utility of whole-exome and transcriptome sequencing by providing a readily available method for rapid interpretation.

It will be appreciated that a variety of similarity metrics may be computed to determine the similarity between cancers of different patients. For example, an individual patient may be actively compared to a larger cohort to return the most similar patient(s). This may be done by turning mutations into a vector for all samples and comparing the similarity of vectors.

Distance within a vector space may be determined in a variety of ways. Euclidean distance, is a simple measure of the straight-line distance between two points in Euclidean space. Euclidean distance has the advantage of simplicity and ease of interpretation. However, it is highly sensitive to noise and outliers along a single dimension, especially for sparse data. In addition, data must be mapped to numeric values.

$$Distance = \sqrt{{\sum }_{i = 1}^{n} {( {q}_{i} - {p}_{i} )}^{2}} \qquad Equation 1$$

Cosine similarity is a measure of angular distance between two points in an n-dimensional space. Cosine similarity works well when there are many features and performs well with sparse data. However, it does not consider the magnitude of point location and is less optimal with a smaller number of features. In addition, data must be mapped to numeric values.

$$similarity = cos ( θ ) = \frac{A \cdot B}{‖ A ‖ ‖ B ‖} \qquad Equation 2$$

Jaccard similarity is defined as the intersection over union between two sets. Data does not have to be mapped to numeric values. Jaccard similarity performs well with sparse data, and works well well with data that has binary attributes. Accordingly, it works well with presence or absence of mutation as a feature. However, it does not perform on real-valued vectors.

$$J ( A , B ) = \frac{\vert A ⋂ B \vert}{\vert A ⋃ B \vert} = \frac{\vert A ⋂ B \vert}{\vert A \vert + \vert B \vert - \vert A ⋂ B \vert} \qquad Equation 3$$

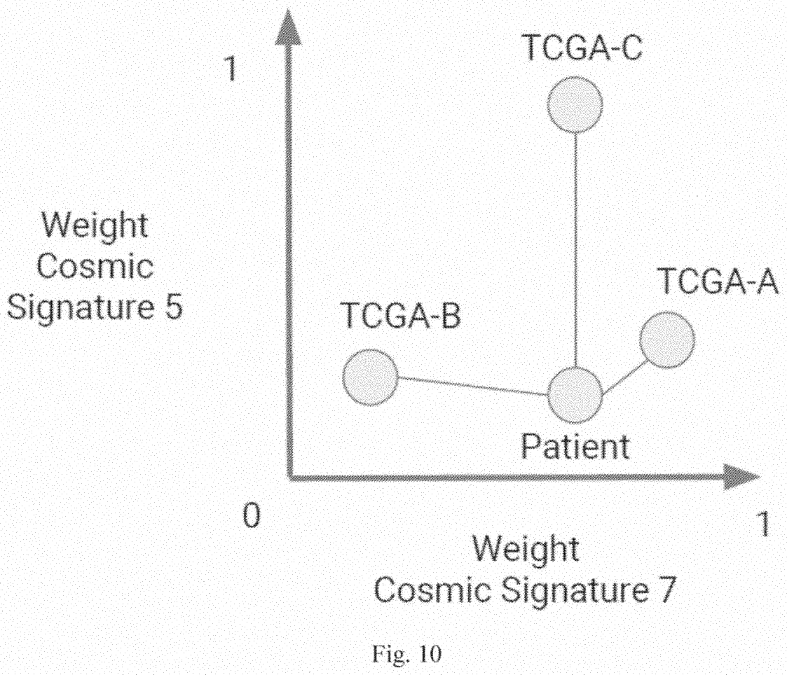

Referring to FIG. 10, an example of Euclidian distance to compute similarity is provided. The Euclidean distance between a patient sample and all points is calculated, returning a ranked list of all samples along this axis. As pictured, the ranked list of all samples returned is: [TCGA-A, TCGA-B, TCGA-C].

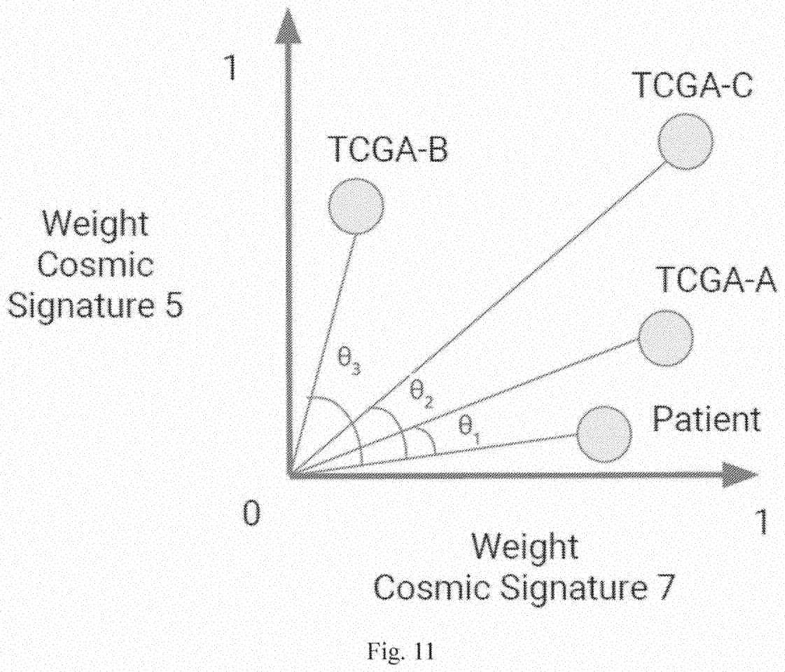

Referring to FIG. 11, an example of cosine distance to compute similarity is provided. The angular distance between the patient sample and all points will be calculated with a cosine similarity, returning a ranked list of all samples along this axis. As pictured, the ranked list of all samples returned is: [TCGA-A, TCGA-C, TCGA-8].

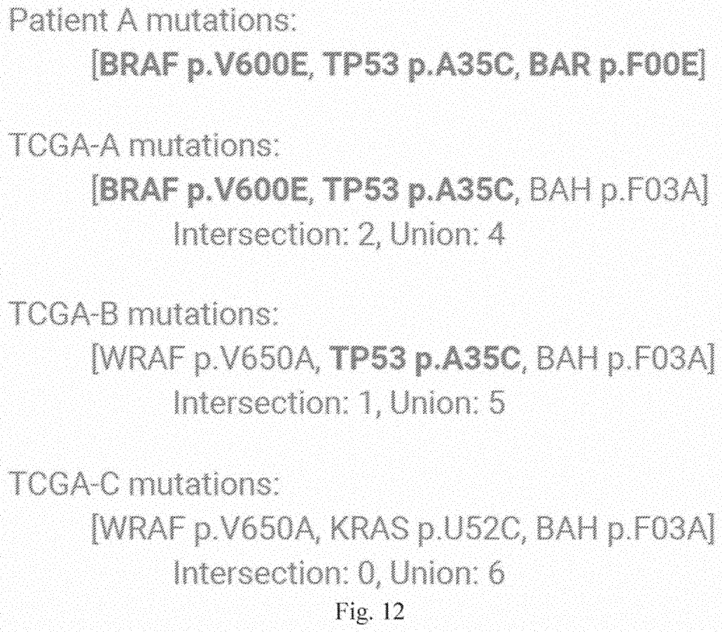

Referring to FIG. 12, an example of Jaccard similarity to compute similarity is provided. The intersection and union of feature sets are calculated for all pairwise relationships relative to the patient samples. The intersection is then divided by the union to calculate the metric. As pictured, the ranked list of all samples returned is: [TCGA-A, TCGA-B, TCGA-C].

Similarity may be considered in terms of global similarity or local similarity. For example, with respect to global similarity, two samples might have 10 of the same genes mutated and 3 of the same contributing mutational signatures. As an example of local similarity, two samples might both have BRAF V600E and significant contribution from COSMIC signature 7 and both don't have KRAS mutations. A suitable solution to match patients needs to incorporate both. A third dimension is also available—population similarity. As an example, the progression mean survival of a given patient might be within a certain std dev of the cluster.

Consider the example of ranking similarity across several features (e.g., Cancer Hotspots, Mutational Signatures, etc.). All vectors are sorted from most similar to least similar. Since all vectors will have as many elements as the comparison cohort, but sorted differently, the Euclidean distance from a patient may be computed.

For example, examining two features and two TCGA classes could yield Euclidian distances as follow:
Feature X: {0:TCGA-A, 1:TCGA-8, 2:TCGA-C, 3:TCGA-D}
Feature Y: {0:TCGA-C, 1:TCGA-A 2:TCGA-8, 3:TCGA-D}
Distance TCGA-A: SQRT(0\hat{ }2+1\hat{ }2)=1
Distance TCGA-8: SQRT(1\hat{ }2+2\hat{ }2)=2.24
Distance TCGA-C: SQRT(2\hat{ }2+0\hat{ }2)=2
Distance TCGA-0: SQRT(3\hat{ }2+3\hat{ }2)=4.25

The above approach only provides a metric for global similarity between patients. Local features may be factored in as well. For example, an additional feature may be added that can show the status of any putatively actionable variants or features. Such a feature may be binary or integer. This addresses the property of Euclidean distance overweighting outliers along a single feature.

For example, adding BRAF V600E could yield Euclidian distances as follow
Feature X: {0:TCGA-A, 1:TCGA-8, 2:TCGA-C, 3:TCGA-D}
Feature Y: {0:TCGA-C, 1:TCGA-A 2:TCGA-8, 3:TCGA-D}
BRAF V600E: {0:TCGA-B, 0:TCGA-C, 3:TCGA-A, 3:TCGA-D}
Distance TCGA-A: SQRT(0\hat{ }2+0\hat{ }2+3\hat{ }2)=3.16
Distance TCGA-B: SQRT(1\hat{ }2+2\hat{ }2+3\hat{ }2)=2.37
Distance TCGA-C: SQRT(2\hat{ }2+1\hat{ }2+2\hat{ }2)=2
Distance TCGA-D: SQRT(3\hat{ }2+3\hat{ }2+0\hat{ }2)=5.20

The above model would give significant weight to chosen local features. As shown, TCGA-A moved from a distance of 1 to 3.16 due to a lack of BRAFV600E. This is important to weight towards individual genomic features. If chosen local features were weighted with respect to cohort size, a heuristic based on putative actionability could be useful. However, the weight should not cause samples that have matching putatively actionable features to fall behind those that do not have any.

Consider a cohort of size n, weights for putative actionability could be as follows:
Putatively Actionable: n/4
Investigate Actionability High: n/2
Investigate Actionability Low: 3n/2
Biologically Relevant: n

The scale for matching is analogous to considering a match for BRAF p.V600E
Putatively Actionable: n/4—BRAF Mutation Missense p.V600E
Investigate Actionability High: n/2—BRAF Mutation Missense p. NSOOA
Investigate Actionability Low: 3n/2—BRAF Mutation Nonsense p.FOOH
Biologically Relevant: n—BRAF CNA Amplification 2.42

In various embodiments, a similarity metric is computed as follows. Jaccard similarity is taken between putatively actionable molecular features, as identified by a molecular oncology almanac, for each level of actionability of an individual sample relative to a cohort. Euclidean distance is taken between individual sample and cohort in R³⁰, where vector space is the contribution of each COSMIC mutational signature. A ranked list of similarity across each feature is then consolidated into an IV space, where Euclidean distance is taken from the origin for each sample in the comparison cohort.

In an exemplary embodiments, samples are selected from an atlas that have whole-exome mutational and copy number data (e.g., 8775 individual tumor samples from TCGA and 418 from CCLE). All samples are analyzed with the Molecular Oncology Almanac to observe putative actionability across all samples and compute mutational signature profile for each sample. Similarity metric is computed pairwise to observe intracohort similarity of TCGA and CCLE cohorts. Similarity metric is computed pairwise of all samples in TCGA to CCLE to generate a null distribution of similarity distances. The similarity metric of individual patient samples is applied to CCLE. The observed distances are compared to that of TCGA-CCLE.

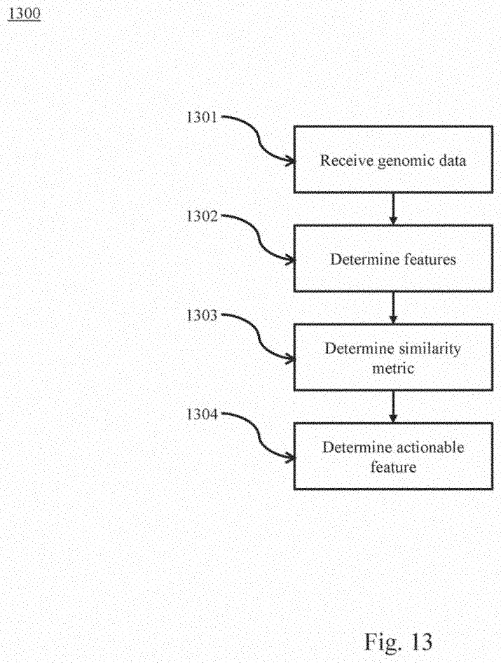

Referring to FIG. 13, a method for feature-based clinical interpretation of genomic data is illustrated. At 1301, genomic data of a subject is received. The genomic data comprises somatic mutations. At 1302, a plurality of features is determined from the genomic data of the subject. At 1303, a similarity metric is determined between the plurality of features and each of a plurality of reference genomes. At 1304, one or more potentially actionable feature is determined from the similarity.

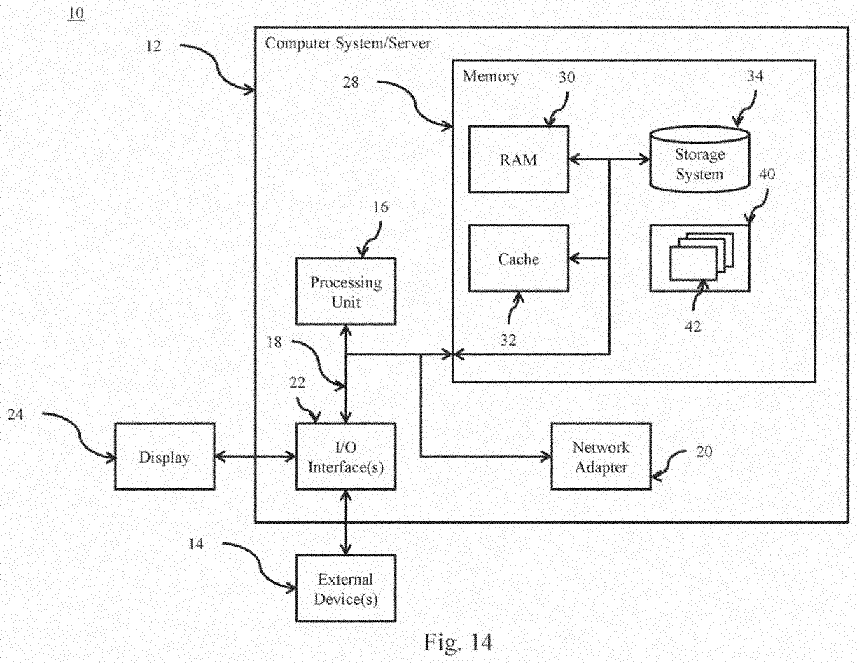

Referring now to FIG. 14, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 13, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
receiving genomic data of a subject, the genomic data comprising somatic mutations;
determining from the genomic data of the subject a plurality of features;
determining a similarity metric between the plurality of features and each of a plurality of reference genomes;
determining from the similarity one or more potentially actionable feature.

2. The method of claim 1, wherein the genomic data of the subject further comprise germline mutations.

3. The method of claim 1, wherein the genomic data of the subject further comprise copy number alterations.

4. The method of claim 1, wherein the genomic data of the subject further comprise fusions.

5. The method of claim 1, further comprising:
determining an associated score for the one or more potentially actionable feature, the score being indicative of support for a clinical action.

6. The method of claim 1, wherein the reference genomes comprise the Cancer Genome Atlas (TCGA).

7. The method of claim 1, wherein the similarity metric comprises a distance within a vector space between a vector corresponding to the plurality of features and vectors corresponding to the plurality of reference genomes.

8. The method of claim 7, wherein the distance comprises a Euclidian distance.

9. The method of claim 7, wherein the distance comprises a cosine distance.

10. The method of claim 7, wherein the distance comprises a Jaccard similarity.

11. The method of claim 1, wherein the plurality of features comprise somatic-germline overlap, DNA-RNA overlap, mutational burden, MSI status, and/or connections.

12. The method of claim 1, wherein the genomic data of the subject is received at a point of care.

13. A system comprising:
a computing node comprising a computer readable storage medium having program instructions embodied therewith, the program instructions executable by a processor of the computing node to cause the processor to perform a method comprising:
receiving genomic data of a subject, the genomic data comprising somatic mutations;
determining from the genomic data of the subject a plurality of features;
determining a similarity metric between the plurality of features and each of a plurality of reference genomes;
determining from the similarity one or more potentially actionable feature.

14. The system of claim 13, wherein the genomic data of the subject further comprise germline mutations.

15. The system of claim 13, wherein the genomic data of the subject further comprise copy number alterations.

16. The system of claim 13, wherein the genomic data of the subject further comprise fusions.

17. The system of claim 13, further comprising:
determining an associated score for the one or more potentially actionable feature, the score being indicative of support for a clinical action.

18. The system of claim 13, wherein the reference genomes comprise the Cancer Genome Atlas (TCGA).

19. The system of claim 13, wherein the similarity metric comprises a distance within a vector space between a vector corresponding to the plurality of features and vectors corresponding to the plurality of reference genomes.

20. The system of claim 19, wherein the distance comprises a Euclidian distance.

21. The system of claim 19, wherein the distance comprises a cosine distance.

22. The system of claim 19, wherein the distance comprises a Jaccard similarity.

23. The system of claim 13, wherein the plurality of features comprise somatic-germline overlap, DNA-RNA overlap, mutational burden, MSI status, and/or connections.

24. The system of claim 13, wherein the genomic data of the subject is received at a point of care.

25. A computer program product for feature-based clinical interpretation of genomic data, the computer program product comprising a computer readable storage medium having program instructions embodied therewith, the program instructions executable by a processor to cause the processor to perform a method comprising:
receiving genomic data of a subject, the genomic data comprising somatic mutations;
determining from the genomic data of the subject a plurality of features;
determining a similarity metric between the plurality of features and each of a plurality of reference genomes;
determining from the similarity one or more potentially actionable feature.

26. The computer program product of claim 25, wherein the genomic data of the subject further comprise germline mutations.

27. The computer program product of claim 25, wherein the genomic data of the subject further comprise copy number alterations.

28. The computer program product of claim 25, wherein the genomic data of the subject further comprise fusions.

29. The computer program product of claim 25, the method further comprising:
determining an associated score for the one or more potentially actionable feature, the score being indicative of support for a clinical action.

30. The computer program product of claim 25, wherein the reference genomes comprise the Cancer Genome Atlas (TCGA).

31. The computer program product of claim 25, wherein the similarity metric comprises computing a distance within a vector space between a vector corresponding to the plurality of features and vectors corresponding to the plurality of reference genomes.

32. The computer program product of claim 31, wherein the distance comprises a Euclidian distance.

33. The computer program product of claim 31, wherein the distance comprises a cosine distance.

34. The computer program product of claim 31, wherein the distance comprises a Jaccard similarity.

35. The computer program product of claim 25, wherein the plurality of features comprise somatic-germline overlap, DNA-RNA overlap, mutational burden, MSI status, and/or connections.

36. The computer program product of claim 25, wherein the genomic data of the subject is received at a point of care.
